(19) United States
(12) Patent Application Publication
(10) Pub. No.: US 2009/0175770 A1
(43) Pub. Date: Jul. 9, 2009

(54) Auto-Lampe portable

(76) Inventor: Maurice Fodera, San Antonio, TX (US)

Correspondence Address: Maurice Fodera, 12435 Panloa Way, San Antonio, TX, 78253, US

(21) Appl. No.: 11/970,090

(22) Filed: Jan. 7, 2008

(51) Int. Cl. B01J 7/00; A61L 9/015; F23Q 7/00

(52) U.S. Cl. 422/306; 239/34; 219/268

(57) ABSTRACT

A catalyst air purifying, fragrance dispenser for a vehicle, such as an automobile, truck or bus, utilizing the vehicle's 12 volt power outlet. The system requires the catalyst to reach 500 degree fahrenheit, the volatile fuel that is also available with a fragrance, is emitted all the while eliminating smoke, unwanted odors and killing airborne bacteria.

The system is refillable by way of exchanging receptacle, or by refilling after fuel is expanded. The top portion has a “grill” allowing air to circulate therefore emitting fragrance and killing bacteria and eliminating smoke and odors.

### BACKGROUND OF THE INVENTION

1. Field of the Invention

This invention relates to a devise for dispensing fragrance or aroma in an automobile or similar vehicle using a 12 volt power source in order to create heat in order for the catalyst ceramic to clean, purify and fragrance the air within. Device will operate for controlled amount of time and is refillable with various fuels, with or without fragrance.

2. Description of Prior Art

The use of air fresheners, air fragrance dispensing devices, and aromatic devices is well known in many environments. Devices are known for providing a fragrance within and automobile, U.S. Pat Des. No. 306,644, issued to Latley Mar. 13, 1990, shows an electric fragrance dispenser for insertion into an automobile cigarette lighter socket.

U.S. Pat No. 5,060,864, issued to Nishi et al. Oct. 29, 1981, shows perfume emitting device for use in an automobile. The Nishi et al. device shows a liquid dispenser that is atomized under pressure carried in a tank located in the vehicle. U.S. Pat. Des. No. 306,644 is a small, portable device that is inserted into a cigarette lighter. One of the drawbacks of the Design Patent item is that, because of the total enclosure of the fragrance, it is impossible to determine the amount of material expanded without disassembling the entire devise.

U.S. Pat. No. 4,814,212, issued to Spector Mar. 21, 1989, shows an automobile air freshener unit that employs a plurality of thin films, a display object, and pressure sensitive adhesive to attach the thin film to a window. An outer film has a fragrance dispensing layer of a volatile fragrance.

U.S. Pat. No. 5,394,506, Issued to Stein Feb. 28, 1995, Shows a see through device that emits fragrance into the air by way of a cigarette lighter power source. This device does not use a catalyst, or a ceramic to purify the air. If not for fragrance this device would be of no use.

### SUMMARY OF THE INVENTION

This invention relates to a device for dispensing fragrance or aroma in an automobile, truck or similar vehicle using the vehicle's 12 volt power source for heating a ceramic catalyst that burns a fuel that rids the air of smoke, unwanted odors, mold and kills 68% of airborne bacteria. This device uses a fuel that has a fragrance and is available with the afore mentioned benefits without fragrance. The device includes a housing, a pair of electrical contacts connected to the housing, with the portion of the housing being cylindrically shaped for fitting into the female socket of a typical vehicle 12 volt power outlet, receiving an electrical current from the 12 volt power outlet system, and a fragrance material storage chamber that includes a ceramic catalyst and a specific heating element alloy placed near or in contact with a vessel containing an air purifying, odor, smoke, mold and airborne bacteria eliminating fuel that burns once operating temperature is achieved through heating element. The device may or may not include an on/off switch, depending on the utilization of the device, or timing device to switch device off after operating temperature s achieved.

The fragrance emitting material, which s in liquid form and contains a volatile or aromatic chemical, is within the device vessel that fits snugly into the overall housing. In a typical embodiment, the air purifying, fragrance emitting material may be of various shapes and sizes to accommodate style and preference. The vessel material is clear and see through and is a separate housing that snaps, in one embodiment, into the basic housing of the overall heating catalyst housing.

The actual material or chemicals used in the air purifying/fragrance dispensing is one with such properties as previously mentioned, is available on the market and is of a particular brand, that which is currently used in homes to produce said results once catalyst is heated using a flame, (purposely absent in this invention.)

What is claimed is:

1. An air purifying, fragrance emitting devise for use in an automobile or other similar type vehicle for dispensing fragrance throughout ambient by way of 12 volt electrical outlet connected to the vehicle electrical power source battery.

2. A first cylindrical housing sized and shaped to be received into a conventional automobile 12 volt power source. Circuit means connected to and routed through said cylindrical housing and adapted to cooperate with circuit elements and heating element alloy for providing heat. A see through material for containing said fuel is of various shapes and sizes to accommodate a catalyst ceramic stone and said fuels in liquid form in order to observe when refill is necessary.

3. A device as in claim a, including means for switching said element on and off, connected to said cylindrical housing and said electrical circuit.

4. A device as in claim b, a particular gage of alloy material as a heating element is inserted in said catalyst ceramic in order to obtain said heating temperature necessary to “burn” said fuels.
